(19) United States
(12) Patent Application Publication
(10) Pub. No.: US 2009/0291982 A1
(43) Pub. Date: Nov. 26, 2009

(54) New Substituted Oxindole Derivative 352

(75) Inventors: Martina Claesson, Sodertalje (SE); Suzanne IVERSON HEMBERG, Sodertalje (SE); Fredrik Lake, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 12/125,619

(22) Filed: May 22, 2008

(51) Int. Cl. A61K 31/4439; C07D 401/04; A61P 25/28; A61P 3/10; A61P 19/10; A61P 19/08

(52) U.S. Cl. 514/339; 546/277.7

(57) ABSTRACT

The present invention relates to a new compound of formula (I)

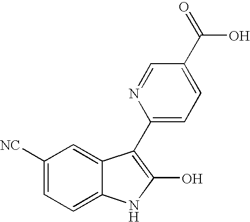

6-(5-cyano-2-hydroxy-1H-indol-3-yl)pyridine-3-carboxylic acid or a pharmaceutically acceptable salt thereof, in an essentially pure and isolated form, pharmaceutical formulations containing said compounds, to the use of said active compounds in therapy, and methods of prevention and/or treatment of conditions associated with glycogen synthase kinase-3 related disorders, comprising administering to a mammal, including human in need of such prevention and/or treatment, a therapeutically effective amount of said compound, as well as a process for preparing said compound.

### FIELD OF THE INVENTION

The present invention relates to a new compound of formula (I) or a pharmaceutically acceptable salt thereof, in an essentially pure and isolated form, to pharmaceutical formulations containing said compounds and to the use of said compounds in therapy. The present invention further relates to a process for the preparation of a compounds of formula (I).

### BACKGROUND OF THE INVENTION

Glycogen synthase kinase 3 (GSK3) is a serine/threonine protein kinase composed of two isoforms (α and β), which are encoded by distinct genes but are highly homologous within the catalytic domain. GSK3 is highly expressed in the central and peripheral nervous system. GSK3 phosphorylates several substrates including tau, β-catenin, glycogen synthase, pyruvate dehydrogenase and elongation initiation factor 2b (eIF2b). Insulin and growth factors activate protein kinase B, which phosphorylates GSK3 on serine 9 residue and inactivates it.

Alzheimer's Disease (AD) Dementias, and Taupathies.

AD is characterized by cognitive decline, cholinergic dysfunction and neuronal death, neurofibrillary tangles and senile plaques consisting of amyloid-β deposits. The sequence of these events in AD is unclear, but is believed to be related. Glycogen synthase kinase 3β (GSK3β) or Tau phosphorylating kinase selectively phosphorylates the microtubule associated protein Tau in neurons at sites that are hyperphosphorylated in AD brains. Hyperphosphorylated tau has lower affinity for microtubules and accumulates as paired helical filaments, which are the main components that constitute neurofibrillary tangles and neuropil threads in AD brains. This results in depolymerization of microtubules, which leads to dying back of axons and neuritic dystrophy. Neurofibrillary tangles are consistently found in diseases such as AD, amyotrophic lateral sclerosis, parkinsonism-dementia of Gaum, corticobasal degeneration, dementia pugilistica and head trauma, Down's syndrome, postencephalatic parkinsonism, progressive supranuclear palsy, Niemann-Pick's Disease and Pick's Disease. Addition of amyloid-β to primary hippocampal cultures results in hyperphosphorylation of tau and a paired helical filaments-like state via induction of GSK3β activity, followed by disruption of axonal transport and neuronal death (Imahori and Uchida, J. Biochem. 1997, 121:179-188). GSK3β preferentially labels neurofibrillary tangles and has been shown to be active in pre-tangle neurons in AD brains. GSK3 protein levels are also increased by 50% in brain tissue from AD patients. Furthermore, GSK3β phosphorylates pyruvate dehydrogenase, a key enzyme in the glycolytic pathway and prevents the conversion of pyruvate to acetyl-Co-A (Hoshi et al., PNAS 1996, 93: 2719-2723). Acetyl-Co-A is critical for the synthesis of acetylcholine, a neurotransmitter with cognitive functions. Accumulation of amyloid-β is an early event in AD. GSK Tg mice show increased levels of amyloid-β in brain. Also, PDAPP mice fed with Lithium show decreased amyloid-β levels in hippocampus and decreased amyloid plaque area (Su et al., Biochemistry 2004, 43: 6899-6908). Thus, GSK3β inhibition may have beneficial effects in progression as well as the cognitive deficits associated with Alzheimer's disease and other above-referred to diseases.

Chronic and Acute Neurodegenerative Diseases

Growth factor mediated activation of the PI3K/Akt pathway has been shown to play a key role in neuronal survival. The activation of this pathway results in GSK3β inhibition. Recent studies (Bhat et. al., PNAS 2000, 97: 11074-11079) indicate that GSK3β activity is increased in cellular and animal models of neurodegeneration such as cerebral ischemia or after growth factor deprivation. For example, the active site phosphorylation was increased in neurons vulnerable to apoptosis, a type of cell death commonly thought to occur in chronic and acute degenerative diseases such as cognitive disorders, Alzheimer's Disease, Parkinson's Disease, amyotrophic lateral sclerosis, Huntington's Disease and HIV dementia and traumatic brain injury; and as in ischemic stroke. Lithium was neuroprotective in inhibiting apoptosis in cells and in the brain at doses that resulted in the inhibition of GSK3β. Thus GSK3β inhibitors could be useful in attenuating the course of neurodegenerative diseases.

Bipolar Disorders (BD)

Bipolar Disorders are characterised by manic episodes and depressive episodes. Lithium has been used to treat BD based on its mood stabilising effects. The disadvantage of lithium is the narrow therapeutic window and the danger of overdosing that can lead to lithium intoxication. The discovery that lithium inhibits GSK3 at therapeutic concentrations has raised the possibility that this enzyme represents a key target of lithium's action in the brain (Stambolic et al., Curr. Biol. 1996, 68(12):1664-1668, 1996; Klein and Melton; PNAS 1996, 93:8455-8459; Gould et al., Neuropsychopharmacology, 2005, 30:1223-1237). GSK3 inhibitor has been shown to reduce immobilisation time in forced swim test, a model to assess on depressive behavior (O'Brien et al., J Neurosci 2004, 24(30): 6791-6798). GSK3 has been associated with a polymorphism found in bipolar II disorder (Szczepankiewicz et al., Neuropsychobiology. 2006, 53: 51-56). Inhibition of GSK3 P may therefore be of therapeutic relevance in the treatment of BD as well as in AD patients that have affective disorders.

Schizophrenia

Accumulating evidence implicates abnormal activity of GSK3 in mood disorders and schizophrenia. GSK3 is involved in signal transduction cascades of multiple cellular processes, particularly during neural development. (Kozlovsky et al., Am. J. Psychiatry, 2000, 157, 5: 831-833) found that GSK3β levels were 41% lower in the schizophrenic patients than in comparison subjects. This study indicates that schizophrenia involves neurodevelopmental pathology and that abnormal GSK3 regulation could play a role in schizophrenia. Furthermore, reduced β-catenin levels have been reported in patients exhibiting schizophrenia (Cotter et al., Neuroreport 1998, 9(7):1379-1383). Atypical antipsychotic such as olanzapine, clozapine, quetiapine, and ziprasidone, inhibits GSK3 by increasing ser9 phosphorylation suggesting that antipsychotics may exert their beneficial effects via GSK3 inhibition (Li X. et al., Int. J.of Neuropsychopharmacol, 2007, 10: 7-19, Epubl. May 4, 2006).

Diabetes

Insulin stimulates glycogen synthesis in skeletal muscles via the dephosphorylation and thus activation of glycogen synthase. Under resting conditions, GSK3 phosphorylates and inactivates glycogen synthase via dephosphorylation. GSK3 is also over-expressed in muscles from Type II diabetic patients (Nikoulina et al., Diabetes February 2000; 49(2): 263-71). Inhibition of GSK3 increases the activity of glycogen synthase thereby decreasing glucose levels by its conversion to glycogen. In animal models of diabetes, GSK3 inhibitors lowered plasma glucose levels up to 50% (Cline et al., Diabetes, 2002, 51: 2903-2910; Ring et al., Diabetes 2003, 52: 588-595). GSK3 inhibition may therefore be of therapeutic relevance in the treatment of Type I and Type II diabetes and diabetic neuropathy.

Alopecia

GSK3 phosphorylates and degrades β-catenin. β-catenin is an effector of the pathway for keratonin synthesis. β-catenin stabilisation may be lead to increase hair development. Mice expressing a stabilised β-catenin by mutation of sites phosphorylated by GSK3 undergo a process resembling de novo hair morphogenesis (Gat et al., Cell, 1998, 95(5): 605-14)). The new follicles formed sebaceous glands and dermal papilla, normally established only in embryogenesis. Thus GSK3 inhibition may offer treatment for baldness.

Inflammatory Disease

The discovery that GSK3 inhibitors provide anti-inflammatory effects has raised the possibility of using GSK3 inhibitors for therapeutic intervention in inflammatory diseases. (Martin et al., Nat. Immunol. 2005, 6(8): 777-784; Jope et al., Neurochem. Res. 2006, DOI 10.1007/s11064-006-9128-5)). Inflammation is a common feature of a broad range of conditions including Alzheimer's Disease and mood disorders.

Cancer

GSK3 is overexpressed in ovarian, breast and prostate cancer cells and recent data suggests that GSK3b may have a role in contributing to cell proliferation and survival pathways in several solid tumor types. GSK3 plays an important role in several signal transduction systems which influence cell proliferation and survival such as WNT, PI3 Kinase and NFkB. GSK3b deficient MEFs indicate a crucial role in cell survival mediated NFkB pathway (Ougolkov A V and Billadeau D D., Future Oncol. February 2006; 2(1): 91-100.). Thus, GSK3 inhibitors may inhibit growth and survival of solid tumors, including pancreatic, colon and prostate cancer.

Bone-Related Disorders and Conditions

GSK3 inhibitors could be used for treatment of bone-related disorders or other conditions, which involves a need for new and increased bone formation. Remodeling of the skeleton is a continuous process, controlled by systemic hormones such as parathyroid hormone (PTH), local factors (e.g. prostaglandin E2), cytokines and other biologically active substances. Two cell types are of key importance: osteoblasts (responsible for bone formation) and osteoclasts (responsible for bone resorption). Via the RANK, RANK ligand and osteoprotegerin regulatory system these two cell types interact to maintain normal bone turnover (Bell N H, Current Drug Targets—Immune, Endocrine & Metabolic Disorders, 2001, 1:93-102).

Osteoporosis is a skeletal disorder in which low bone mass and deterioration of bone microarchitecture lead to increased bone fragility and fracture risk. To treat osteoporosis, the two main strategies are to either inhibit bone resorption or to stimulate bone formation. The majority of drugs currently on the market for the treatment of osteoporosis act to increase bone mass by inhibiting osteoclastic bone resorption. It is recognized that a drug with the capacity to increase bone formation would be of great value in the treatment of osteoporosis as well as having the potential to enhance fracture healing in patients.

Recent in vitro studies suggest a role of GSK3β in osteoblast differentiation. First, it has been shown that glucocorticoids inhibit cell cycle progression during osteoblast differentiation in culture. The mechanism behind this is activation of GSK3β in osteoblasts, resulting in c-Myc down-regulation and impediment of the G₁/S cell cycle transition. The attenuated cell cycle and reduced c-Myc level are returned to normal when GSK3β is inhibited using lithium chloride (Smith et al., J. Biol. Chem, 2002, 277: 18191-18197). Secondly, inhibition of GSK3β in the pluripotent mesenchymal cell line C3H10T1/2 leads to a significant increase in endogenous β-catenin signaling activity. This, in turn, induces expression of alkaline phosphatase mRNA and protein, a marker of early osteoblast differentiation (Bain et al., Biochem. Biophys. Res. Commun., 2003, 301: 84-91).

### DETAILED DESCRIPTION OF THE INVENTION

The object of the present invention is to provide a new compound having a selective inhibiting effect at GSK3 as well as having a good bioavailability. Accordingly, the present invention provides a compound of formula (I)

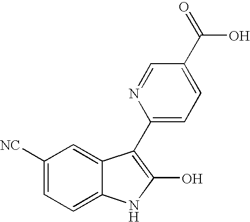

6-(5-cyano-2-hydroxy-1H-indol-3-yl)pyridine-3-carboxylic acid or a pharmaceutically acceptable salt thereof, in an essentially pure and isolated form.

The compounds of formula (I), 6-(5-cyano-2-hydroxy-1H-indol-3-yl)pyridine-3-carboxylic acid has been identified as a metabolite of 2-hydroxy-3-[5-(morpholin-4-ylmethyl)pyridin-2-yl]1H-indole-5-carbonitrile in rat, dog and/or man in in-vivo studies.

Thus, a further object of the present invention is a method of using 2-hydroxy-3-[5-(morpholin-4-ylmethyl)pyridin-2-yl]1H-indole-5-carbonitrile to administer a metabolite of formula (I), the 6-(5-cyano-2-hydroxy-1H-indol-3-yl)pyridine-3-carboxylic acid or a pharmaceutically acceptable salt thereof, in an essentially pure and isolated form.

Thus, an object of the present invention is a metabolite compound of formula (I) when prepared ex-vivo.

The compound 2-hydroxy-3-[5-(morpholin-4-ylmethyl)pyridin-2-yl]1H-indole-5-carbonitrile is disclosed in WO 03/082853.

The present invention also relates to the use of a compound of formula (I) as hereinbefore defined.

Salts for use in pharmaceutical formulations will be pharmaceutically acceptable salts, but other salts may be useful in the production of the compounds of formula (I).

Pharmaceutical Formulations

According to one aspect of the present invention there is provided a pharmaceutical formulation comprising the compound of formula (I) or a pharmaceutically acceptable salt thereof, in an essentially pure and isolated form, for use in the prevention and/or treatment of conditions associated with glycogen synthase kinase-3.

The formulation used in accordance with the present invention may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration as an ointment, patch or cream, for rectal administration as a suppository and for local administration in a body cavity or in a bone cavity.

The formulation may be in a form suitable for oral administration, for example as a tablet, for parenteral injection as a sterile solution or suspension. In general the above formulation may be prepared in a conventional manner using pharmaceutically carriers or diluents.

Suitable daily doses of the compound of formula (I) or pharmaceutically acceptable salts thereof in the treatment of a mammal, including human, are approximately 0.01 to 250 mg/kg bodyweight at per oral administration and about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

The compound of formula (I) or a pharmaceutically acceptable salt thereof, in an essentially pure and isolated form, may be used on its own but will usually be administered in the form of a pharmaceutical formulation in which the active ingredient is in association with pharmaceutically acceptable diluents, excipients or inert carrier. Dependent on the mode of administration, the pharmaceutical formulation may comprise from 0.05 to 99% w (per cent by weight), for example from 0.10 to 50% w, of active ingredient, all percentages by weight being based on total composition.

A diluent or carrier includes water, aqueous poly(ethylene glycol), magnesium carbonate, magnesium stearate, talc, a sugar (such as lactose), pectin, dextrin, starch, tragacanth, microcrystalline cellulose, methyl cellulose, sodium carboxymethyl cellulose or cocoa butter.

A formulation of the invention can be in a unit dosage form such as a tablet or an injectable solution. The tablet may additionally comprise a disintegrant and/or may be coated (for example with an enteric coating or coated with a coating agent such as hydroxypropyl methylcellulose).

The invention further provides a process for the preparation of a pharmaceutical formulation of the invention which comprises mixing of the compound of formula (I) or a pharmaceutically acceptable salt thereof, a hereinbefore defined, with pharmaceutically acceptable diluents, excipients or inert carriers.

An example of a pharmaceutical formulations of the invention is an injectable solution comprising the compound of formula (I) or a pharmaceutically acceptable salt thereof, as hereinbefore defined, and sterile water, and, if necessary, either a base or an acid to bring the pH of the final formulation to a pH in the range of about 4 to 6, particularly about 5, and optionally a surfactant to aid dissolution. A suitable base is sodium hydroxide. A suitable acid is hydrochloric acid.

A suitable pharmaceutically acceptable salt of the compound of formula (I) useful in accordance to the invention is, for example, an acid-addition salt, which is sufficiently basic, for example an inorganic or organic acid. In addition a suitable pharmaceutically acceptable salt of the compounds of the invention, which is sufficiently acidic, is an alkali metal salt, an alkaline earth metal salt or a salt with an organic base, which affords a physiologically-acceptable cation.

Medical Uses

It has been found that the compound of formula (I) defined in the present invention, are well suited for inhibiting glycogen synthase kinase-3 (GSK3). Accordingly, said compound of the present invention is expected to be useful in the prevention and/or treatment of conditions associated with glycogen synthase kinase-3 activity, i.e. the compounds may be used to produce an inhibitory effect of GSK3 in mammals, including human, in need of such prevention and/or treatment.

GSK3 is highly expressed in the central and peripheral nervous system and in other tissues. Thus, it is expected that compound of the invention is well suited for the prevention and/or treatment of conditions associated with glycogen synthase kinase-3 in the central and peripheral nervous system. In particular, the compound of the invention is expected to be suitable for prevention and/or treatment of conditions associated with cognitive disorders and predemented states, especially dementia, Alzheimer's Disease (AD), Cognitive Deficit in Schizophrenia (CDS), Mild Cognitive Impairment (MCI), Age-Associated Memory Impairment (AAMI), Age-Related Cognitive Decline (ARCD) and Cognitive Impairement No Dementia (CIND), diseases associated with neurofibrillar tangle pathologies, Frontotemporal dementia (FTD), Frontotemporal dementia Parkinson's Type (FTDP), progressive supranuclear palsy (PSP), Pick's Disease, Niemann-Pick's Disease, corticobasal degeneration (CBD), traumatic brain injury (TBI) and dementia pugilistica.

One embodiment of the invention relates to the prevention and/or treatment of Alzheimer's Disease, especially the use in the delay of the disease progression of Alzheimer's Disease.

Other conditions are selected from the group consisting of Down's syndrome, vascular dementia, Parkinson's Disease (PD), postencephelatic parkinsonism, dementia with Lewy bodies, HIV dementia, Huntington's Disease, amyotrophic lateral sclerosis (ALS), motor neuron diseases (MND, Creuztfeld-Jacob's disease and prion diseases.

Other conditions are selected from the group consisting of attention deficit disorder (ADD), attention deficit hyperactivity disorder (ADHD) and affective disorders, wherein the affective disorders are Bipolar Disorder including acute mania, bipolar depression, bipolar maintenance, major depressive disorders (MDD) including depression, major depression, mood stabilization, schizoaffective disorders including schizophrenia, and dysthymia.

Other conditions are selected from the group consisting of Type I diabetes, Type II diabetes, diabetic neuropathy, alopecia, inflammatory diseases and cancer.

One embodiment of the invention relates to the use of a compound of the formula (I), as defined in the present invention, in the prevention and/or treatment of bone-related disorders or conditions in mammals.

One aspect of the invention is directed to the use of a compound of the formula (I), as defined in the present invention to treat osteoporosis.

One aspect of the invention is directed to the use of a compound of the formula (I), as defined in the present invention to increase and promote bone formation in mammals.

One aspect of the invention is directed to the use of a compound of the formula (I), as defined in the present invention to increase bone mineral density in mammals.

Another aspect of the invention is directed to the use of a compound of the formula (I), as defined in the present invention to reduce the rate of fracture and/or increase the rate of fracture healing in mammals.

Another aspect of the invention is directed to the use of a compound of the formula (I), as defined in the present invention to increase cancellous bone formation and/or new bone formation in mammals.

Another aspect of the invention is directed to a method of prevention and/or treatment of bone-related disorders comprising administering to a mammal in need of such prevention and/or treatment, a therapeutically effective amount of a compound of the formula (I) as defined in the present invention.

Another aspect of the invention is directed to a method of prevention and/or treatment of osteoporosis comprising administering to a mammal in need of such prevention and/or treatment, a therapeutically effective amount of a compound of the formula (I) as defined in the present invention.

Another aspect of the invention is directed to a method of increasing bone formation comprising administering to a mammal in need of such treatment, a therapeutically effective amount of a compound of the formula (I) as defined in the present invention.

Another aspect of the invention is directed to a method of increasing bone mineral density comprising administering to a mammal in need of such treatment, a therapeutically effective amount of a compound of the formula (I) as defined in the present invention.

Another aspect of the invention is directed to a method of reducing the incidence of fracture comprising administering to a mammal in need of such treatment, a therapeutically effective amount of a compound of the formula (I) as defined in the present invention.

Another aspect of the invention is directed to a method of enhancing fracture healing comprising administering to a mammal in need of such treatment, a therapeutically effective amount of a compound of the formula (I) as defined in the present invention.

Another aspect of the invention is directed to said methods and wherein said mammal is a human.

Another aspect of the invention is directed to said methods and wherein said mammal is a vertibrate animal, preferably but not limited to bigger animals such as horses, camels, dromedars but not limited thereto.

The use of the GSK3 inhibitors, the compounds of formula (I) hereinbefore defined, in primary and secondary ostopeorosis, where primary osteoporosis includes postmenopausal osteoporosis and senile osteoporosis in both men and women, and secondary osteoporosis includes cortison induced osteoporosis, as well as any other type of induced secondary osteoporosis, are included in the term osteoporosis. In addition to this, these GSK3 inhibitors may also be used in treatments of myeloma. These GSK3 inhibitors may be administered locally or systemically, in different formulation regimes, to treat these conditions.

The promotion and increasing of bone formation and/or bone mineral density makes the compound of the formula (I) hereinbefore defined, suitable to reducing the incidence of fracture, to reduce the rate of fracture and/or increase the rate of fracture healing, to increase cancellous bone formation and/or new bone formation in mammals.

The use to promote and increase new bone formation may be in connection with surgery. This invention can be used during surgery, where the treating surgeon will place the invention locally in an appropriate formulation, near the deficient bone and/or in the body cavity. The bone may for instance have been broken, and utilizing the invention as described and claimed herein will then be placed in or near the fracture during open fracture repair. In some instances bone pieces may be missing (e.g. after tumour removal or severe casualties), and utilizing the invention as described and claimed herein will then be placed near the site of constructive bone surgery.

The present invention relates also to the use of the compound of formula (I) as as defined in the present invention in the manufacture of a medicament for the prevention and/or treatment of conditions associated with glycogen synthase kinase-3.

The invention also provides for a method of treatment and/or prevention of conditions associated with glycogen synthase kinase-3 comprising administering to a mammal, including human in need of such treatment and/or prevention a therapeutically effective amount of the compound of formula (I) as as defined in the present invention.

The dose required for the therapeutic or preventive treatment of a particular disease will necessarily be varied depending on the host treated, the route of administration and the severity of the illness being treated.

For veterinary use the amounts of different components, the dosage form and the dose of the medicament may vary and will depend on various factors such as, for example the individual requirement of the animal treated.

In the context of the present specification, the term “therapy” also includes “prevention” unless there are specific indications to the contrary. The terms “therapeutic” and “therapeutically” should be construed accordingly.

In the context of the present specification, the term “disorder” also includes “condition” unless there are specific indications to the contrary.

Another aspect of the invention is wherein a compound of formula (I) or a pharmaceutically acceptable salt thereof asa defied herein, or a pharmaceutical composition or formulation comprising such a compound of formula (I) is administered concurrently, simultaneously, sequentially or separately with another pharmaceutically active compound or compounds selected from the following:

(i) antidepressants such as agomelatine, amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, ramelteon, reboxetine, robalzotan, sertraline, sibutramine, thionisoxetine, tranylcypromaine, trazodone, trimipramine, venlafaxine and equivalents and pharmaceutically active isomer(s) and metabolite(s) thereof.

(ii) atypical antipsychotics including for example quetiapine and pharmaceutically active isomer(s) and metabolite(s) thereof.

(iii) antipsychotics including for example amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutylpiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents and pharmaceutically active isomer(s) and metabolite(s) thereof.

(iv) anxiolytics including for example alnespirone, azapirones,benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents and pharmaceutically active isomer(s) and metabolite(s) thereof.

(v) anticonvulsants including for example carbamazepine, valproate, lamotrogine, gabapentin and equivalents and pharmaceutically active isomer(s) and metabolite(s) thereof.

(vi) Alzheimer's therapies including for example donepezil, memantine, tacrine and equivalents and pharmaceutically active isomer(s) and metabolite(s) thereof.

(vii) Parkinson's therapies including for example deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase and equivalents and pharmaceutically active isomer(s) and metabolite(s) thereof.

(viii) migraine therapies including for example almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents and pharmaceutically active isomer(s) and metabolite(s) thereof.

(ix) stroke therapies including for example abciximab, activase, NXY-059, citicoline, crobenetine, desmoteplase,repinotan, traxoprodil and equivalents and pharmaceutically active isomer(s) and metabolite(s) thereof.

(x) urinary incontinence therapies including for example darafenacin, falvoxate, oxybutynin, propiverine, robalzotan, solifenacin, tolterodine and and equivalents and pharmaceutically active isomer(s) and metabolite(s) thereof.

(xi) neuropathic pain therapies including for example gabapentin, lidoderm, pregablin and equivalents and pharmaceutically active isomer(s) and metabolite(s) thereof.

(xii) nociceptive pain therapies such as celecoxib, etoricoxib, lumiracoxib, rofecoxib, valdecoxib, diclofenac, loxoprofen, naproxen, paracetamol and equivalents and pharmaceutically active isomer(s) and metabolite(s) thereof.

(xiii) insomnia therapies including for example agomelatine, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, ramelteon, roletamide, triclofos,secobarbital, zaleplon, zolpidem and equivalents and pharmaceutically active isomer(s) and metabolite(s) thereof.

(xiv) mood stabilizers including for example carbamazepine, divalproex, gabapentin, lamotrigine, lithium, olanzapine, quetiapine, valproate, valproic acid, verapamil, and equivalents and pharmaceutically active isomer(s) and metabolite(s) thereof.

Such combination products employ the compound of this invention within the dosage range described herein and the other pharmaceutically active compound or compounds within approved dosage ranges and/or the dosage described in the publication reference.

Methods of Preparation

The present invention also relates to a process for preparing the compound of formula (I)

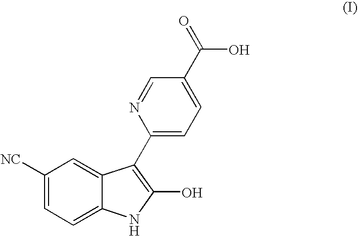

6-(5-cyano-2-hydroxy-1H-indol-3-yl)pyridine-3-carboxylic acid or a pharmaceutically acceptable salt thereof, in an essentially pure and isolated form, which comprises:

A process for preparing the compound of formula (I), by

ai) reacting a compound of formula (II), with a compound of formula (A), where Hal is halogen, e.g. fluorine, chlorine or bromine and R¹ is C1-6 alkyl, e.g. ethyl, in the presence of a base under inert atmosphere, e.g. under argon athmosphere. A suitable base may be an alkali metal hydride such as lithium hydride or sodium hydride, an organic amine base such as pyridine, 2,6-lutidine, collidine, 4-dimethylaminopyridine, triethylamine, morpholine, N-methylmorpholine, diazabicyclo[5.4.0]undec-7-ene, tetramethylguanidine or an alkaline earth metal carbonate such as sodium carbonate, potassium carbonate or calcium carbonate. Alternatively, such a base may be an alkali metal or an alkaline earth metal amide such as sodium amide, sodium bis(trimethylsilyl)amide, potassium amide or potassium bis(trimethylsilyl)amide. The reaction may be carried out in an appropriate solvent such as an ether e.g. tetrahydrofuran or 1,4-dioxan, an aromatic hydrocarbon solvent e.g. toluene, or a dipolar aprotic solvent e.g, N,N-dimethylformamide, N,N-dimethylacetamide, N-methylpyrrolidin-2-one (most preferred), dimethylsulphoxide or mixtures thereof, the reaction may be carried out at a temperature between +10° C. and +150° C.,

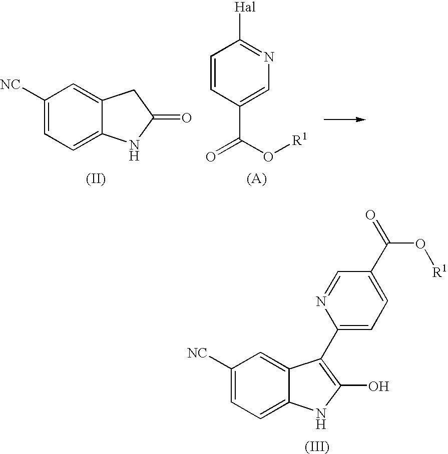

followed by

aii) hydrolysis of a compound of formula (III) to a compound of formula (I). The reaction may be carried out by using an alkali metal hydroxide such as lithium hydroxide, sodium hydroxide, potassium hydroxide or barium hydroxide in an appropriate solvent such as tetrahydrofuran, ethyl ether, 1,4-dioxane, methanol, ethanol, propanol or mixture thereof. The reaction may be carried out at a temperature between +10° C. and +80° C.

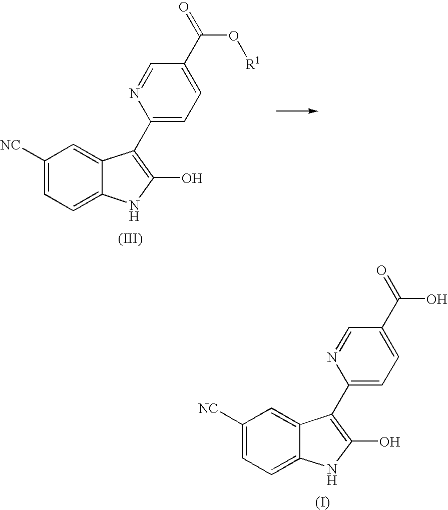

Where necessary converting the resultant compound of formula (I), or another salt thereof, into a pharmaceutically acceptable salt thereof.

One embodiment of the process for preparing the compound of formula (I) is, by

ai) reacting a compound of formula (II), with a compound of formula (A), where Hal is fluorine, chlorine or bromine and R¹ is C1-6 alkyl, e.g. as ethyl, in the presence of lithium hydride as a base under argon athmosphere at a temperature between +10° C. and +80° C., followed by

aii) hydrolysis of the obtained compound of formula (III) to a compound of formula (I) using lithium hydroxide in a mixture of tetrahydrofuran and ethanol at a temperature between +10° C. and +80° C.

Starting materials used such as 2-oxindoline-5-carbonitrile (II) and a compound of formula (A), for example ethyl 6-chloropyridine-3-carboxylate were available from commercial sources, or may be prepared according to literature procedures.

### WORKING EXAMPLES

The following working example will describe, but not limit, the invention.

### Example 1

### 6-(5-cyano-2-hydroxy-1H-indol-3-yl)pyridine-3-carboxylic acid

### (a) Ethyl 6-(5-cyano-2-hydroxy-1H-indol-3-yl)pyridine-3-carboxylate

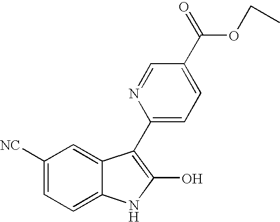

Lithium hydride (51 mg, 6.10 mmol, 95%) was added to 2-oxo-1,3-dihydroindole-5-carbonitrile (0.48 g, 3.05 mmol) in NMP (5.0 mL) under argon atmosphere. The mixture was flushed with argon and ethyl 6-chloropyridine-3-carboxylate (0.85 g, 4.58 mmol) was added dropwise. The mixture was heated at 50° C. for 1 h and additional ethyl 6-chloropyridine-3-carboxylate (0.28 g, 1.53 mmol) was added. The mixture was heated at 75° C. for 3 h and allowed to cool over night, and was poured into a mixture of NH₄Cl (sat.) and EtOAc. The aqeous phase was extracted with EtOAc and was filtered. The yellow/orange solids (0.14 mg, 0.46 mmol, 15%) were dried in a 40° C. vacuum oven over night.

1H NMR (400 MHz, DMSO-d₆) δ 8.73 (s, 1 H) 7.96 (br. s., 2 H) 7.79 (d, 1 H) 7.37 (d, 1 H) 7.02 (d, 1 H) 4.30 (q, 2 H) 1.30 (t, 3 H); MS (ESI) m/z 308 (M+1).

### (b) 6-(5-cyano-2-hydroxy-1H-indol-3-yl)pyridine-3-carboxylic acid

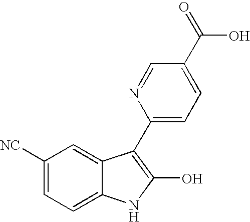

A solution of LiOH monohydrate (77 mg, 1.82 mmol) in water (1.5 mL) was added to ethyl 6-(5-cyano-2-hydroxy-1H-indol-3-yl)pyridine-3-carboxylate (140 mg, 0.46 mmol) in THF (2.5 mL) and EtOH (1.5 mL). The mixture was stirred at ambient temperature for 4.5 h and was purified by prepHPLC. Freeze-drying of the combined fractions gave the title compound as a bright yellow acetate salt (14 mg, 0.050 mmol, 11%).

1H NMR (MeOH-d₄) δ 8.56 (d, 1 H) 8.34 (dd, 1 H) 7.91 (d, 1 H) 7.74 (d, 1 H) 7.31 (dd, 1 H) 7.11 (d, 1 H) 1.91 (s, 3 H); MS (ESI) m/z 278 (M−1).

General Methods

¹H NMR spectra were recorded in the indicated deuterated solvent at 400 MHz or 500 MHz. The 400 MHz spectra were obtained using a Bruker av400 NMR spectrometer equipped with a 3 mm flow injection SEI ¹H/D-¹³C probe head with Z-gradients, using a BEST 215 liquid handler for sample injection, or using a Bruker DPX400 NMR or Bruker 500 MHz ultrashield spectrometer equipped with a 4-nucleus probehead with Z-gradients. Chemical shifts are given in ppm down- and upfield from TMS. Resonance multiplicities are denoted s, d, t, q, m and br for singlet, doublet, triplet, quartet, multiplet, and broad respectively.

LC-MS analyses were recorded on a Waters LCMS equipped with a Waters X-Terra MS, C8-column, (3.5 □m, 100 mm×3.0 mm i.d.). The mobile phase system consisted of A: 10 mM ammonium acetate in water/acetonitrile (95:5) and B: acetonitrile. A linear gradient was applied running from 0% to 100% B in 4-5 minutes with a flow rate of 1.0 mL/min. The mass spectrometer was equipped with an electrospray ion source (ESI) operated in a positive or negative ion mode. The capillary voltage was 3 kV and the mass spectrometer was typically scanned between m/z 100-700. Alternative, LC-MS HPLC conditions were as follows: Column: Agilent Zorbax SB-C8 ( 5 □m, 50 mm×2 mm i.d) Flow: 1.0 mL/minGradient: 95% A to 100% B in 5 min. A=5% acetonitrile in water with 0.1% formic acid and B=acetonitrile with 0.1% formic acid. UV-DAD 210-400 nm Alternative, LC-MS analyses were recorded on a Waters 2790 LCMS equipped with a Phenomenex Luna C18 (5 □m, 50×4.6 mm i.d.) The mobile phase system consisted of A: 10 mM ammonium formate( pH 4) in water and B: acetonitrile. A linear gradient was applied running from 95% to 5% B in 5 minutes with a flow rate of 2.0 mL/min. The mass spectrometer was equipped with an electrospray ion source (ESI) operated in a positive or negative ion mode. The capillary voltage was 3 kV and the mass spectrometer was typically scanned between m/z 100-700.

Mass spectra (MS) were run using an automated system with atmospheric pressure chemical (APCI or CI) or electrospray (+ESI) ionization. Generally, only spectra where parent masses are observed are reported. The lowest mass major ion is reported for molecules where isotope splitting results in multiple mass spectral peaks (for example when chlorine is present).

HPLC assays were performed using an Agilent HP 1100 Series system equipped with a Waters X-Terra MS, C₈ column (3.0×100 mm, 3.5 μm). The column temperature was set to 40° C. and the flow rate to 1.0 mL/min. The Diode Array Detector was scanned from 200-300 nm. A linear gradient was applied, run from 0% to 100% B in 4 min. Mobile phase A: 10 mM ammonium acetate in water/acetonitrile (95:5), mobile phase B: acetonitrile.

HPLC purities were performed using a Dionex P680 Series system equipped with a Genesis AQ, (100×4.6 mm, 4 μm) column. The column temperature was set to 25° C. and the flow rate to 1.5 mL/min. The Diode Array Detector was scanned from 200-300 nm. The mobile phase system comprise of A: 10/90 (v/v) acetonitrile/phosphate buffer (25 mM, pH 6.8) and B: 70/30 (v/v) acetonitrile/phosphate buffer (25 mM, pH 6.8). A gradient was applied according to the table below:

| Time (mm) | % B |
|---|---|
| 0 | 5 |
| 5 | 5 |
| 20 | 100 |
| 21 | 5 |
| 25 | 5 |

Preparative HPLC was performed on a Waters Auto purification HPLC-UV system with a diode array detector using a Waters XTerra® MS C₈ column (19×300 mm, 7 μm) with the gradient described.

The compound have been named using ACD/Name, version 8.08, software from Advanced Chemistry Development, Inc. (ACD/Labs), Toronto ON, Canada, www.acdlabs.com, 2004 or are according to IUPAC convention.

Pharmacology

Determination of ATP Competition in Scintillation Proximity GSK3β Assay.

GSK3β Scintillation Proximity Assay.

The competition experiments were carried out in duplicate with 10 different concentrations of the inhibitors in clear-bottom microtiter plates (Wallac, Finland). A biotinylated peptide substrate, Biotin-Ala-Ala-Glu-Glu-Leu-Asp-Ser-Arg-Ala-Gly-Ser(PO₃H₂)-Pro-Gln-Leu (AstraZeneca, Lund), was added at a final concentration of 1 μM in an assay buffer containing 1 mU recombinant human GSK3β (Dundee University, UK), 12 mM morpholinepropanesulfonic acid (MOPS), pH 7.0, 0.3 mM EDTA, 0.01% β-mercaptorethanol, 0.004% Brij 35 (a natural detergent), 0.5% glycerol and 0.5 μg BSA/25 μl. The reaction was initiated by the addition of 0.04 μCi[γ-³³P]ATP (Amersham, UK) and unlabelled ATP at a final concentration of 1 μM and assay volume of 25 μl. After incubation for 20 minutes at room temperature, each reaction was terminated by the addition of 25 μl stop solution containing 5 mM EDTA, 50 μM ATP, 0.1% Triton X-100 and 0.25 mg streptavidin coated Scintillation Proximity Assay (SPA) beads (Amersham, UK). After 6 hours the radioactivity was determined in a liquid scintillation counter (1450 MicroBeta Trilux, Wallac). The inhibition curves were analysed by non-linear regression using GraphPad Prism, USA. The Kₘ value of ATP for GSK3β, used to calculate the inhibition constants (Kᵢ) of the various compounds, was 20 βM.

The following abbreviations have been used:
MOPS Morpholinepropanesulfonic acid
EDTA Ethylenediaminetetraacetic acid
BSA Bovin Serum Albumin
ATP Adenosine Triphosphate
SPA Scintillation Proximity Assay
GSK3 Glycogen synthase kinase 3

Results

The Kᵢ value for the compound of formula (I) of the present invention is 32 nM.

1. A compound of formula (I) 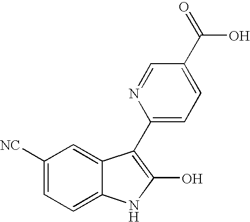
6-(5-cyano-2-hydroxy-1H-indol-3-yl)pyridine-3-carboxylic acid or a pharmaceutically acceptable salt thereof, in an essentially pure and isolated form.

2. A pharmaceutical formulation comprising as active ingredient a therapeutically effective amount of the compound according to claim 1, optionally in association with diluents, excipients or inert carriers.

3. A pharmaceutical formulation according to claim 2 in the form of an injectable solution comprising the compound of formula (I) as defined in claim 1, sterile water, and, if necessary, either a base, particularly sodium hydroxide, or an acid, particularly hydrochloric acid, to bring the pH of the final formulation to a pH in the range of about 4 to 6, and optionally a surfactant.

4. An injectable solution according to claim 3, where the pH is about 5.

5. A compound as defined in claim 1 for use in therapy.

6. A method of using 2-hydroxy-3-[5-(morpholin-4-ylmethyl)pyridin-2-yl]1H-indole-5-carbonitrile to administer a metabolite of formula (I), the 6-(5-cyano-2-hydroxy-1H-indol-3-yl)pyridine-3-carboxylic acid or a pharmaceutically acceptable salt thereof, in an essentially pure and isolated form.

7. A method of prevention and/or treatment of conditions associated with glycogen synthase kinase-3, comprising administering to a mammal, including human in need of such prevention and/or treatment, a therapeutically effective amount of a compound as defined in claim 1.

8. A method of prevention and/or treatment of cognitive disorders, comprising administrering to a mammal, including human in need of such prevention and/or treatment, a therapeutically effective amount of a compound as defined in claim 1.

9. The method according to claim 8, wherein the cognitive disorder is dementia, Cognitive Deficit in Schizophrenia (CDS), Mild Cognitive Impairment (MCI), Age-Associated Memory Impairment (AAMI), Age-Related Cognitive Decline (ARCD) or Cognitive Impairement No Dementia (CIND).

10. The method according to claim 9, wherein the disease is Cognitive Deficit in Schizophrenia.

11. The method according to claim 9, wherein the dementia is associated with neurofibrillar tangle pathologies.

12. The method according to claim 9, wherein the dementia is Frontotemporal dementia (FTD), Frontotemporal dementia Parkinson's Type (FTDP), progressive supranuclear palsy (PSP), Pick's Disease, Niemann-Pick's Disease, corticobasal degeneration, traumatic brain injury (TBI) or dementia pugilistica.

13. The method according to claim 9, wherein the dementia is Alzheimer's Disease (AD), Down syndrome, vascular dementia, Parkinson's Disease (PD), postencephelatic parkinsonism, dementia with Lewy bodies, HIV dementia, Huntington's Disease, amyotrophic lateral sclerosis (ALS), motor neuron diseases (MND), Creuztfeld-Jacob's disease or prion diseases.

14. The method according to claim 13, wherein the disease is Alzheimer's Disease.

15. The method according to claim 14, wherein the treatment is in the delay of the disease progression of Alzheimer's Disease.

16. A method of prevention and/or treatment of attention deficit disorder (ADD), attention deficit hyperactivity disorder (ADHD) or affective disorders, comprising administrering to a mammal, including human in need of such prevention and/or treatment, a therapeutically effective amount of a compound as defined in of claim 1.

17. The method according to claim 16, wherein the affective disorders are Bipolar Disorder including acute mania, bipolar depression, bipolar maintenance, major depressive disorders (MDD) including depression, major depression, mood stabilization, schizoaffective disorders including schizophrenia, or dysthymia.

18. A method of prevention and/or treatment of Type I diabetes, Type II diabetes, diabetic neuropathy, alopecia, inflammatory diseases or cancer, comprising administrering to a mammal, including human in need of such prevention and/or treatment, a therapeutically effective amount of a compound as defined in claim 1.

19. A method of prevention and/or treatment of bone related disorders or conditions comprising administrering to a mammal, including human in need of such prevention and/or treatment, a therapeutically effective amount of a compound as defined in claim 1.

20. A method of prevention and/or treatment of osteoporosis comprising administrering to a mammal, including human in need of such prevention and/or treatment, a therapeutically effective amount of a compound as defined in claim 1.

21. A method of increasing bone formation comprising administrering to a mammal, including human in need of such prevention and/or treatment, a therapeutically effective amount of a compound as defined in claim 1.

22. A method of increasing cancellous bone formation and/or new bone formation comprising administrering to a mammal, including human in need of such prevention and/or treatment, a therapeutically effective amount of a compound as described in claim 1.

23. A method of increasing bone mineral density comprising administrering to a mammal, including human in need of such prevention and/or treatment, a therapeutically effective amount of a compound as defined in claim 1.

24. A method of reducing the incidence of fracture comprising administrering to a mammal, including human in need of such prevention and/or treatment, a therapeutically effective amount of a compound as defined in claim 1.

25. A method of enhancing fracture healing comprising administrering to a mammal, including human in need of such prevention and/or treatment, a therapeutically effective amount of a compound as described in claim 1.

26. A process for preparing the compound of formula (I) according to claim 1, by
ai) reacting a compound of formula (II), with a compound of formula (A), where Hal is halogen and R¹ is C1-6 alkyl, in the presence of a base under inert atmosphere in an appropriate solvent at a temperature between +10° C. and +150° C., 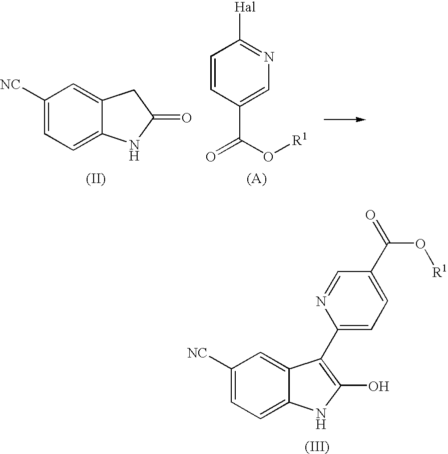
followed by
aii) hydrolysis of the obtained compound of formula (III) to a compound of formula (I) in the prescence of an alkali metal in an appropriate solvent at a temperature range between +10° C. and +80° C. 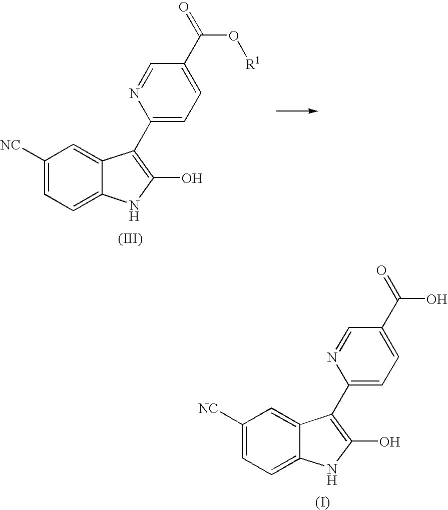
and where necessary converting the resultant compound of formula (I), or another salt thereof, into a pharmaceutically acceptable salt thereof

27. A process according to claim 26, where the base is lithium hydride.
